(19) United States
(12) Patent Application Publication
(10) Pub. No.: US 2008/0139076 A1
(43) Pub. Date: Jun. 12, 2008

(54) Decorative cover for intravenous bag

(75) Inventor: Karen Frances Frasier-Scott, Houston, TX (US)

Correspondence Address: ELIZABETH R. HALL, 1722 MARYLAND STREET, HOUSTON, TX, 77006, US

(73) Assignee: Envirotech Designs, Inc., Houston, TX (US)

(21) Appl. No.: 11/986,492

(22) Filed: Nov. 21, 2007

Related U.S. Application Data
(60) Provisional application No. 60/873,500, filed on Dec. 7, 2006.

(51) Int. Cl. A61B 19/02

(52) U.S. Cl. 446/72

(57) ABSTRACT

A cover for intravenous fluid bags is used to disguise the IV bag thereby reducing the level of patient fear and intimidation created by medical equipment and environments. The decorative cover can be shaped to portray a familiar object (animal, plant, vehicle), or be provided with a blank surface that can accommodate colorful stickers, drawings or removable interchangeable objects selected by the patient. The cover is comprised of an opaque front surface and an attachment means for attaching the cover to the IV bag or the IV stand. The cover is easily attached or removed by medical staff and does not obstruct the normal monitoring of fluid progress from the bag.

### CROSS REFERENCE TO RELATED APPLICATIONS

This application claims priority to pending U.S. patent application Ser. No. 60/873,500 (Attorney Docket Number KFFS-P001V), filed Dec. 7, 2006 by inventor Karen Frances Frasier-Scott and entitled “Life Shield.”

### FIELD OF THE INVENTION

The present invention relates to a covering for intravenous fluid bags used in the delivery of medical therapy to a patient. In particular, the present invention relates to a decorative cover for intravenous bags that when used, will reduce patient stress.

### DESCRIPTION OF THE RELATED ART

Approximately 50,000 children each year in the United States are diagnosed with cancer and are hospitalized for treatment. In addition, several thousand more children are hospitalized for severe illnesses and accidents each year. Statistics provided by the Lucille Packard Children's Hospital at Stanford of Palo Alto, Calif., show that children ages 14 and under account for one-third of all fall-related visits to hospital emergency rooms. In 2003, nearly 285,600 children ages 14 and under were treated in the U.S. for bicycle-related injuries. Nearly half (47 percent) of children ages 14 and under hospitalized for bicycle-related injuries are diagnosed with a traumatic brain injury. The statistics for other child related injuries is staggering. All of these children face a stay in a hospital.

Visits to a hospital can be an intimidating, frightening, and stress producing experience for children. The fear and anxiety of being in a new environment, confronted with new faces and unfamiliar medical instruments, particularly critical care patients undergoing intravenous treatments, can be intimidating and extremely stressful.

The medical equipment in the hospital environment can also be intimidating, in particular tubes running from the child's hand or arm to a myriad of bags hanging from metal poles on stands causes fear in the child because they realize they are going to be hooked up to or attached to some type of medical equipment.

The stress resulting from the unfamiliarity with the surroundings, intimidating environment and intimidating medical equipment can stimulate a sympathetic nervous response. It is well-established that stress-induced sympathetic nervous system responses in patients lead to such objective physical findings as increased heart rate, increased blood pressure, sweating, and emotional outbursts (Guyton, A. C. and J. E. Hall. 1996. Textbook of Medical Physiology, ninth edition. WB Saunders Company: Philadelphia, chapter 60). The stress caused can also inhibit immune system function (Schimmer, B. P. and K. L. Parker. 2006. In: Goodman & Gilman 's The pharmacological Basis of Therapeutics, eleventh edition. Brunton et al (eds), McGraw-Hill: New York, pp. 1591) which could slow and/or hinder recovery, posing an imminent health risk in critically ill children. Illness and injury themselves cause stress in patients, while the burden of being hooked up to or attached to the equipment and its cumbersome immobility causes additional stress.

In research done on the problem of stress related to visualization of the hospital environment, when asked, one adult solution was to place items on the walls of the hospital room for the child to see. Children on the other hand wanted the intravenous bags hidden or protected from view mainly because the bags and stands were in closer proximity and were constantly with the child. While placing items on the walls of the child's hospital room helped to relieve stress by bringing familiar objects in the line of sight of the child, this in and of itself was not a complete solution. This is because when the child has to leave the environment of the hospital room, the intravenous bags and stand have to travel with the child and become a constant reminder that the child is still attached to medical equipment. Hiding the intravenous bag or disguising its presence in a way that the child is familiar with would aid in the reduction of stress. Another advantage of disguising or hiding the intravenous bag while the child is in motion outside the hospital room is the unintentional reduction of stress in other children who may see the disguised or hidden intravenous bag. Instead of focusing on the child's illness or injury, other children will tend to focus on the disguise to the intravenous bag.

The visualization and stress production related to medical equipment has been recognized as a problem and has been addressed in prior art. U.S. Pat. No. 5,592,946 discloses decorative coverings for stethoscopes to inhibit allergic reactions caused by tubing on the instrument as well as to lessen intimidation in young patients. The coverings described are hypoallergenic fabric stethoscope covers with hook-and-loop fasteners for attaching a closure portion of the cover and to affix the cover around the tubing of the stethoscope. Only fabric materials are disclosed or suggested for the decorative covers.

U.S. Pat. Nos. 6,165,035 and 6,520,639 disclose decorative coverings for medical equipment where the exterior of the covering is shaped to portray things such as animals, plants, vehicles, and buildings for use in disguising the various types of medical equipment. Specific embodiments of the invention included coverings for stethoscopes, blood pressure cuffs, otoscopes, opthalmoscopes, blood pressure cuffs, intravenous stands, crutches, and wheelchairs. The coverings are constructed of fabric.

Other patents disclosing decorative covers for medical exam equipment include U.S. Pat. No. 5,038,755 and U.S. Pat. No. 6,095,647. Both patents disclose disguising medical equipment to make the equipment less intimidating to children. In one case, a toy-like object is molded to an otoscope (U.S. Pat. No. 5,038,755) while in the other case a mask is attached to the head of an opthalmoscope that is shaped like an animal or some other familiar object (U.S. Pat. No. 6,095,647).

U.S. Pat. No. 4,934,336 describes an apparatus for warming intravenous fluids in intravenous fluid bags that consists of a wrap material. The wrap material is constructed of insulated nylon and is fixed around the intravenous fluid bag using some type of closure device.

Problems with the prior art, primarily covers placed on or covering the intravenous stands, prove to be expensive and cumbersome and can interfere with medical personnel's monitoring the intravenous bag contents and replacing the intravenous bags and mobility of the stands when outside the hospital room environment.

There is a need for a covering of the intravenous bag from the patient's view.

### SUMMARY OF THE INVENTION

The present invention provides an intravenous bag cover which can be decorated by drawing directly on the cover or by placing decorative stickers directly on the cover, or fabricating the cover in the shape of objects such as animals, birds or vehicles. The cover is of one-piece construction comprised of a moldable elastomeric material with a curved surface conforming to the shape of the intravenous bag it covers.

One embodiment of the present invention is a cover for an intravenous fluid bag comprising: (a) a opaque front surface having a surface area as large as the largest intravenous fluid bag surface; (b) a side panel attached to the front surface substantially perpendicular to the front surface; and (c) a positioning means for positioning the front surface of the cover to block the frontal view of the contents of the intravenous fluid bag.

Another embodiment of the present invention is a cover for an intravenous fluid bag comprising: (a) an opaque front surface of the cover having a surface area as large as the largest surface of the intravenous fluid bag; and (b) two side panels, positioned on opposed edges of the front surface substantially perpendicular to the front surface, wherein the two side panels are rounded to cover two sides of the intravenous fluid bag when the front surface is positioned to cover the intravenous fluid bag surface opposed to the bag surface displaying information on the contents of the intravenous fluid bag; whereby the front surface of the cover blocks the frontal view of the contents of the intravenous fluid bag whenever the cover is positioned in front of the intravenous fluid bag surface opposed to the bag surface displaying information on the contents of the intravenous fluid bag and whereas viewing the information on the contents of the intravenous fluid bag is unhindered by the cover when the cover is so positioned.

Yet another embodiment of the present invention is a cover for an intravenous fluid bag comprising: (a) an opaque front surface having a three sided frame attached thereto, wherein a first side of the frame is proximal a bottom edge of the front surface and a second and a third side of the frame are attached to opposed ends of the first side of the frame; (b) a side panel attached to an edge of the front surface substantially perpendicular to the front surface; and (c) a hook attached to an upper edge of the front surface of the cover; whereby whenever the cover is attached to the intravenous fluid bag pole with the hook, the cover blocks the frontal view of the contents of the intravenous fluid bag.

The present invention also provides a kit comprising the cover, an attachment means for attaching the cover to the intravenous bag or intravenous stand and at least one means for decorating the cover.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 shows three intravenous fluid bags of differing sizes, a 50 ml bag, a 500 ml bag, and a 1000 ml bag suspended on an intravenous pole.

FIG. 2 shows a view of the information side of the intravenous fluid bag and a backside view of a cover attached to the IV bag.

FIG. 3 shows a frontal view of a cover.

FIG. 4 shows an oblique view of a cover with a hand drawn design.

FIG. 5 shows an oblique view of a pre-embossed figure on a cover.

FIG. 6 shows a front view of the cover with a three sided frame attached thereto and a removable plate that fits into the frame.

FIG. 7 shows an oblique view of a three sided embodiment of the cover with a transparent back panel.

FIG. 8 shows an oblique view of a three sided embodiment of the cover where the back panel is a frame.

FIG. 9 shows an oblique view of a cover having a hook attached to a top edge of the cover.

FIGS. 10A-B illustrate frontal and side views of a cover with a swivel hook attached to the cover.

FIG. 11 shows the back view of a cover with Velcro strips attached and a frontal view of an intravenous fluid bag with Velcro strips attached.

FIG. 12 shows one embodiment of a cover kit comprised of a pre-molded cover, a three-sided frame, removable plates, and decorative items such as a sticker and/or coloring materials.

FIG. 13 shows one embodiment of a cover kit comprised of a pre-embossed cover, and coloring materials.

FIG. 14 shows a pre-packaged, pre-sterilized cover.

It is noted that like reference characters designate like or similar parts throughout the drawings. The figures, or drawings, are not intended to be to scale.

### DETAILED DESCRIPTION OF THE INVENTION

The present invention relates to an apparatus and method for covering intravenous (also referred to herein as “IV”) fluid bags used in the delivery of medical therapy to patients. The intravenous (“IV”) fluid cover 10 has a curved surface conforming to the shape of the intravenous bag it covers.

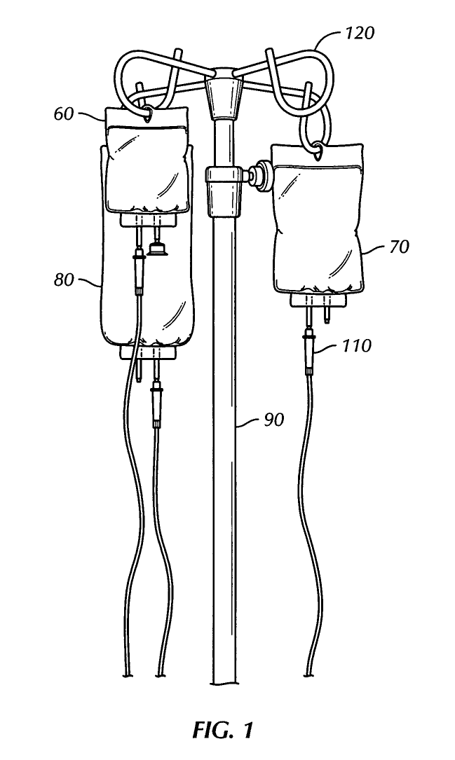

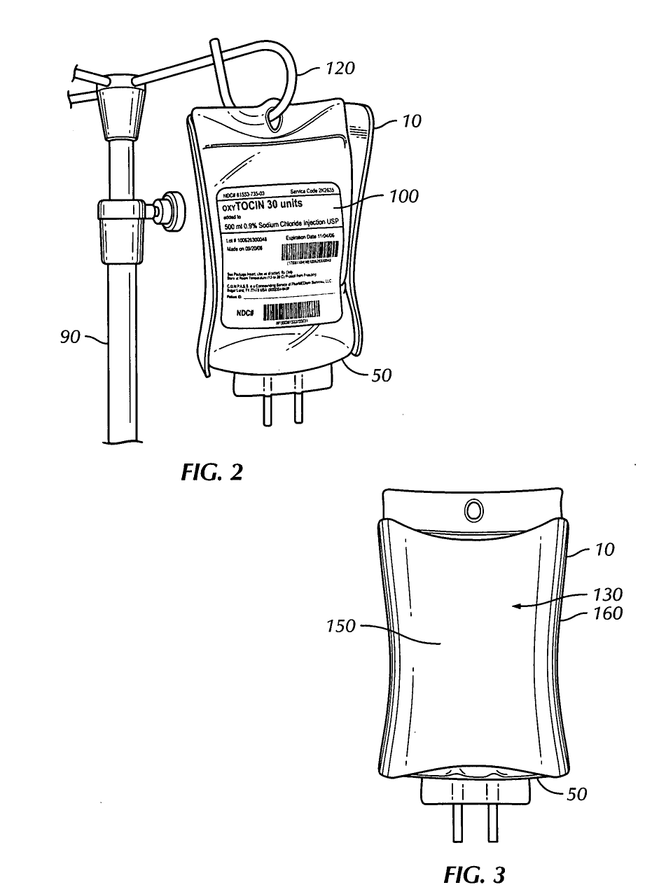

FIG. 2 shows an intravenous (“IV”) fluid bag 50 as used in hospital environments. FIG. 1 shows three intravenous fluid bags of differing sizes, a 50 ml bag 60, a 500 ml bag 70, and a 1000 ml bag 80 suspended on a pole 90. The IV bags are shown with drip lines 110. The top of the pole 90 is capped by “P” shaped hooks 120 from which the intravenous fluid bags are suspended. As shown in FIG. 2, each intravenous fluid bag 50 displays information 100 regarding the contents of the intravenous bag.

The purpose and place of the intravenous fluid bag in patient treatment is multi-functional. The intravenous fluid bags are used to deliver medical therapy to critically ill patients, post-operative patients, and to provide fluids to patients prior to and after surgery.

Often treatments associated with intravenous fluid bags, whether in critically ill patients or pre- and post-operative patients, are directly connected to the patient's fear of pain. The combination of the intravenous fluid bag 50, the drip line 110 and attachment to the patient's arm or hand are all commingled in the patient's mind to induce high levels of stress and fear. This is especially true in critically ill patients.

The question is why cover the intravenous bag at all. Research has proven that patients, particularly children and older adults, can be particularly vulnerable to intimidation by the hospital environment, including unfamiliar medical equipment and medical personnel. The patients may become overly fearful, can be in a highly agitated state of anticipated pain, and are stressed due to an awareness of losing control of their environment. The present invention is designed to assist in alleviating the stress caused by the patient's intimidation by presenting a familiar object, drawing, or photo to cover the intravenous fluid bag, changing the patient's line of sight to a more familiar and comforting environment.

Care must be taken however to insure that the cover can be easily and quickly installed and removed. The cover cannot interfere with the medical personnel's activities to monitor the fluids in the bag or hide the information side of the bag from view. Further, the cover should not inhibit intravenous pole movement to accommodate placement in different locations in the hospital room, moving the pole out of the hospital room, or placing the pole in the patient's home.

The intravenous fluid bag cover 10 is made to cover at least one side of the intravenous fluid bag (i.e., the flat side opposite to the information side of the bag). Thus, the intravenous fluid bag cover 10 may take any shape that fulfills that function. For example, the specific dimensions of the cover 10 will vary depending on the standard dimensions of the size intravenous fluid bag that the cover 10 is designed to cover. Although the cover 10 can be made of any material, a preferred embodiment is made of a moldable elastomeric material that is rigid or semi-rigid. For example, one embodiment of the intravenous fluid bag cover 130, shown in FIG. 3, is a one-piece moldable elastomeric material, such as styrene plastic, which can be sterilized by UV radiation.

The intravenous fluid bag cover 10 typically includes an opaque front surface 150, at least one side panel 160, and a positioning means for positioning the front surface 150 in front of the IV bag to block one side of the IV bag from view. The positioning means typically attaches the cover 10 to the hooks 120 of the pole 90 or to the front surface of the bag 50 opposite the information side of the IV bag. The bag cover 10 must be easy to install on the bag 50 without hiding the information on the bag 50.

The front surface 150, positioned directly in front of a side of the UV bag 50 opposite to the flat side of the IV bag having the information on the contents of the bag 10, is decorated to direct attention from the intravenous fluid bag 50. For example, the cover 130 has a flat surface face 150 and both the right and left edges 160 are rounded and molded to conform to the shape of the bag it covers.

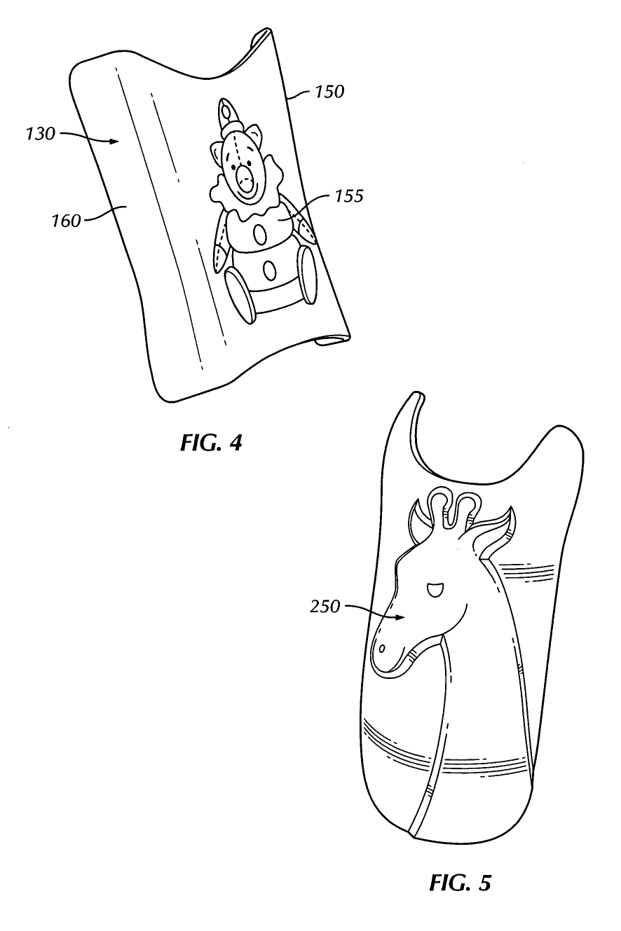

The face or front surface 150 of the cover 10 can be configured in any number of ways, as long as it covers enough surface area on the front of the intravenous fluid bag 50 to prevent the patient's direct observation of the contents of the bag. For example, the cover can comprise a flat, relatively smooth blank front surface 150 that a patient or other person can decorate by applying stickers, prefabricated scenes, magnetic objects, floral arrangements, or animal caricatures. In addition, the patient or caregiver may personalize the front surface 150 with a drawing 155 as shown in FIG. 4.

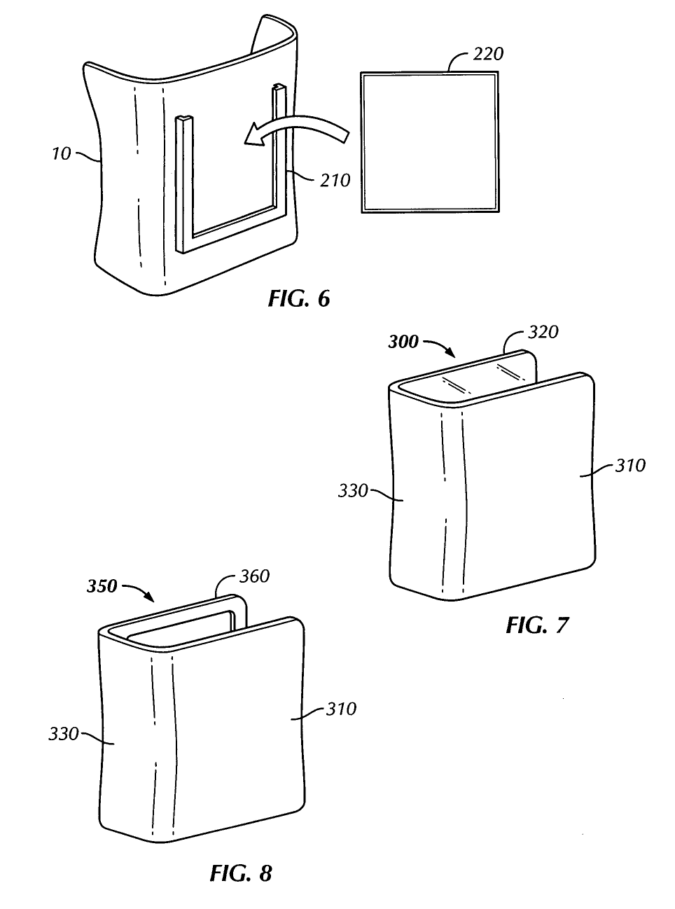

Alternatively, the front surface 150 can have selectably removable plates 220 as illustrated in FIG. 6. To accommodate the removable plates 220, the front surface 150 of the cover 10 may include a frame 210 attached to the front surface 150 which would allow for insertion and removal of the plates 220, or other objects such as a favorite photo, a pre-drawn picture from home, or interchangeable scenic views, sports figures, floral vistas, all of which can be replaced by simply sliding the current plate or representation out and dropping in a new representation. For example, the plates 220 can fit into a three-side pocket-like frame 210 attached to the front surface 150. The pocket-like frame 210 can accommodate photos, prefabricated inserts, or the removable plates 220.

The patient can draw on the plates 220, or the patient can decorate the plates 220 with stickers or other objects. In addition, the plate might be used by hospital personnel to display a patient identifier. Regardless of its use, one removable plate 220 can be interchanged with another decorated plate 220.

Another embodiment of the cover 250, shown in FIG. 5, is a pre-molded material, or an embossed material, having a representation of a familiar object or figure such as an animal, a religious symbol, a vehicle, or cartoon character. The cover 250 can be further embellished by drawing on its surface as well as placing pre-adhesive and pre-designed stickers on it.

Optionally, the cover 10 may include one or two sides 160 attached to the front surface 150 of the cover 10. The sides 160 of the cover 10 may be a variety of configurations. For example, the sides may be rectangular, rounded, oblong, or contoured as seen in FIG. 3. The sides 160 may also be encompassed in part of an embossed design.

The cover 10, whatever its configuration, is made to cover a sufficient portion of the surface area of the front of the intravenous fluid bag 50 to prevent the patient's direct observation of the contents of the bag.

In one embodiment not shown in the figures, the cover is constructed of cloth in the form of a bag that can be attached to the intravenous fluid bag by slipping the cloth bag over the intravenous fluid bag. The rear or back side of the cloth bag would have a clear plastic window for viewing information on the intravenous fluid bag.

In another embodiment shown in FIG. 7, the cover 300 consists of three plates forming a three-sided box structure. A first plate 310 serves as the front surface 150 described above. For example, the first plate 310 my be constructed of an opaque elastomeric material upon which stickers can be applied, photos attached, magnetic objects attached to, or is a blank surface for drawing. The second plate 320 is constructed of a clear or transparent elastomeric material which allows an easy and unhindered view of vital information on the bag. The two plates are fitted together in an approximately parallel fashion by a third plate 330 constructed of an elastomeric material, such that the cover assumes the shape of a 3-sided box so that the bag 50 can slip into the cover via the open side.

FIG. 8 illustrates a cover 350, similar to the cover 300 shown in FIG. 7, except that the second plate 360 is not a solid plate but is a frame where the vital information on the bag 50 is clearly visible in the open area surrounded by the frame of the second plate 360.

In another embodiment the cover 10 is made of a single, molded elastomeric unit that fits around three sides of an intravenous fluid bag as illustrated in FIGS. 3 and 6.

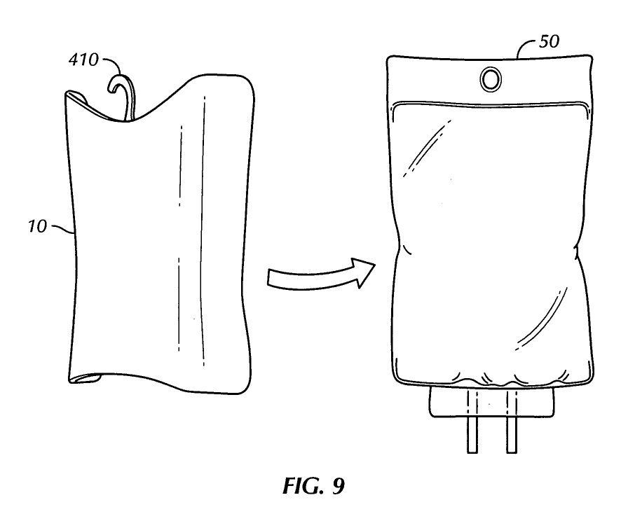

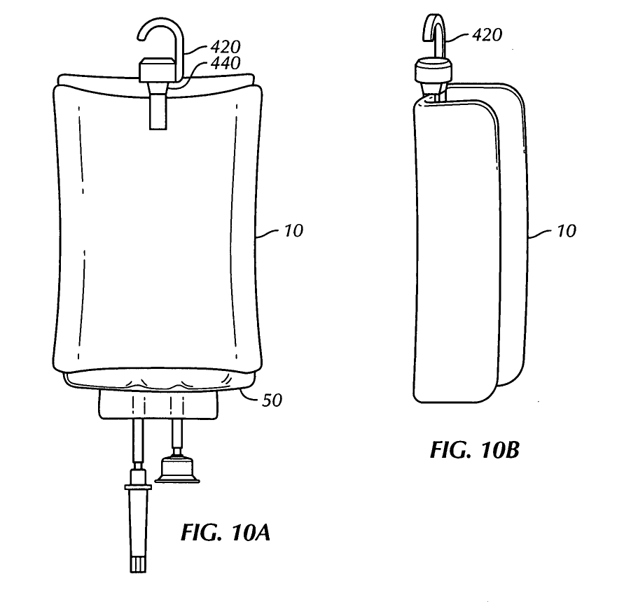

The cover 10 is positioned with an attachment means to the intravenous fluid bag 50 or to the pole 90. A variety of attachment means are suitable for attaching the cover 10 to the bag 50 or the pole 90. For example, the cover 10 may be attached to the pole hook 120 using a simple fixed hook 410, or a flexible hook, attached to the cover 10 as shown in FIG. 9. Similarly, the cover 10 may be attached to the pole hook 120 using a hook 420 attached to a swivel device 440 that allows the cover 10 to be easily swiveled about the pole hook 120 to provide unhindered visual access to the information side of the IV bag 50, as illustrated in FIGS. 10A-B.

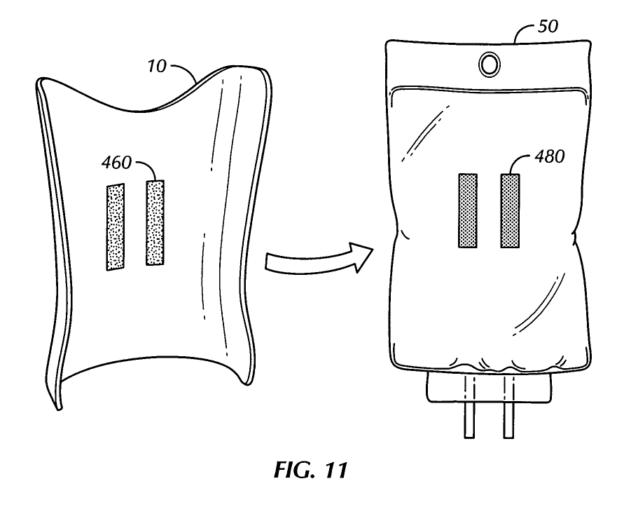

In yet another embodiment, the cover can be attached to the intravenous fluid bag 50 by means of one or more strips 460 of a double-sticky adhesive or a Velcro-like material with hooks and loops, where the cover adhesive strips 460 are placed on the inside of the front surface of the cover 10, and comating adhesive strips 480 are placed on the IV bag 50 as shown in FIG. 11. When the adhesive strips 460 are comated with the strips 480, the cover 10 is adhered to the non-information side of the IV bag 50. In this embodiment, the cover may be constructed as a single flat plate of elastomeric material.

Other methods of attaching the cover to the intravenous fluid bag are also contemplated by the present invention and would include but not be limited to a cloth hanger affixed to the top of the cover, a hole punched into the top of the cover 10 that is large enough for insertion of the hook 120 found at the top of the intravenous pole 90, straps attached to the sides of the cover that can be tied to the intravenous fluid bag 50, or metal clips that can be attached to the cover and clip to the edges of the intravenous fluid bag 50. Any method of affixing a cover to an intravenous fluid bag, without disrupting the flow of fluid or interfering with the monitoring of the fluid and its contents, can be used with the cover 10. The attachment means must also allow for a quick and easy attachment and removal.

The present invention includes a prepackaged sterilized cover 600, where a decorated prepackaged and sterilized cover is selected by the patient and used in environments for the immune impaired patient, such as a patient in a burn unit. The prepackaged sterilized cover 600, shown in FIG. 14, may be sterilized by gamma radiation or any other means.

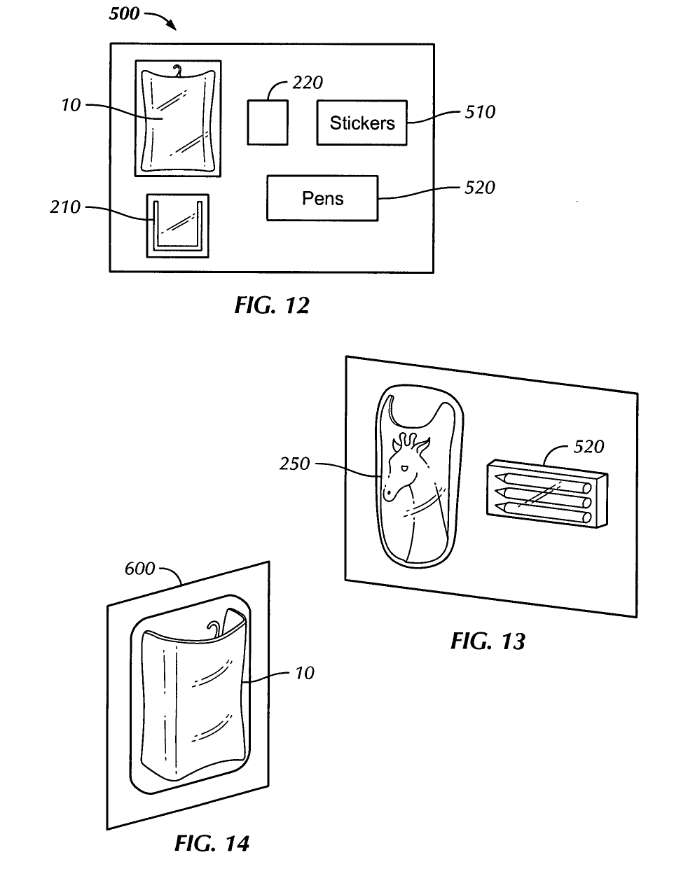

The present invention also includes kits containing a cover 10 and materials for decorating or embellishing the cover (see FIGS. 12 and 13). One embodiment of a kit illustrated in FIG. 12 includes a cover 10, a selectably attachable frame 210, a set of removable plates 220, a pre-adhesive set of stickers or pre-designed objects 510 (representing sports figures, animals, dinosaurs, cartoon characters, or stock photos of animals, vehicles, flowers, or religious icons), and a set of coloring pens 520 for drawing on the front surface 150 of the cover or on the removable plates 220. Another example of a kit comprises a pre-embossed cover 250 and coloring pens 520.

The contents of the kits of the present invention can be given to a patient prior to, or in case of emergency situations after, a medical procedure for decoration or embellishment. This decoration or embellishment makes the overall environment of the intravenous fluid bag and pole less stressful and after application of the decoration, a more familiar and friendlier object.

When in the waiting room, this preparatory embellishment of the cover is less frightening to a patient and reduces the stress level in the patient because he or she is distracted by choosing which stickers to place on the cover or which colors to choose when drawing on the cover. The embellishment of the cover makes it a piece of art or a toy-like item designed by the patient which provides a very familiar object to be placed in the hospital room. The distraction of working on the cover is also an aid to care-giving adults, by focusing their attention on working with the patient to design the cover. The care-giver's level of stress and apprehension will generally diminish as the patient's stress decreases.

A means of disguising an intravenous fluid bag is not only limited to children in a hospital setting. Mentally challenged children in long-term care facilities and adults in nursing care facilities or home care facilities can also benefit from the covers. These patients suffer from stress just as children admitted to hospitals for injuries or illness show stress. Their environments also contain hospital equipment including intravenous bags and stands. The disclosed cover goes beyond any prior art by placing a cover over the intravenous fluid bag thus making the bag look like a familiar object, such as an animal, a vehicle, a sports figure, a bouquet of flowers, a cartoon character, or any other decoration that is applied of a free-hand drawing or embellishment. A pre-fabricated cover with a photograph or previously drawn picture of something personal to the patient is also contemplated as part of the present invention.

While preferred embodiments of the invention have been described herein, many variations thereof will be apparent to those skilled in the art without departing from the spirit of the invention. Accordingly, it is intended that the scope of the invention be limited only by the claims which follow.

What is claimed is:

1. A cover for an intravenous fluid bag comprising:
(a) an opaque front surface having a surface area as large as the intravenous fluid bag surface to be covered;
(b) a side panel attached to the front surface substantially perpendicular to the front surface; and
(c) a positioning means for positioning the front surface of the cover to block the frontal view of the contents of the intravenous fluid bag.

2. The cover of claim 1, wherein the cover is made of an elastomeric material.

3. The cover of claim 1, wherein the cover is sterilized.

4. The cover of claim 1 further comprising a three-sided frame attached to the front surface.

5. The cover of claim 4 further comprising selectably removable plates that fit into the frame.

6. The cover of claim 1, wherein the front surface has a design embossed thereon.

7. The cover of claim 1, wherein the positioning means includes a swivel hook for attaching the cover to a hook proximal to a top of an intravenous fluid bag pole.

8. The cover of claim 1, wherein the positioning means includes an adhesive material removably attaching the cover to the intravenous fluid bag.

9. The cover of claim 8, wherein the adhesive material is Velcro.

10. A cover for an intravenous fluid bag comprising:
(a) an opaque front surface of the cover having a surface area as large as the surface of the intravenous fluid bag to be covered; and
(b) two side panels, positioned on opposed edges of the front surface, wherein the two side panels are rounded to cover two sides of the intravenous fluid bag when the front surface is positioned to cover the intravenous fluid bag surface opposed to the bag surface displaying information on the contents of the intravenous fluid bag;
whereby the front surface of the cover blocks the frontal view of the contents of the intravenous fluid bag whenever the cover is positioned in front of the intravenous fluid bag surface opposed to the bag surface displaying information on the contents of the intravenous fluid bag, and whereas viewing the information on the contents of the intravenous fluid bag is unhindered by the cover when the cover is so positioned.

11. The cover of claim 10, wherein a three sided frame is attached to the front surface of the cover such that a first side of the frame is proximal a bottom edge of the front surface and a second and a third side of the frame are attached to opposed ends of the first side of the frame.

12. The cover of claim 10 further comprising an attachment means for attaching the cover to an intravenous fluid bag pole.

13. The cover of claim 11 wherein the attachment means includes a flexible hook attached to a top edge of the front surface.

14. The cover of claim 12 made of a rigid elastomeric material.

15. The cover of claim 10 further comprising an attachment means for attaching the cover to the intravenous fluid bag.

16. A cover for an intravenous fluid bag comprising:
(a) an opaque front surface having a three sided frame attached thereto, wherein a first side of the frame is proximal to a bottom edge of the front surface and a second and a third side of the frame are attached to opposed ends of the first side of the frame;
(b) a side panel attached to an edge of the front surface substantially perpendicular to the front surface; and
(c) a hook attached to an upper edge of the front surface of the cover;
whereby a frontal view of the contents of the intravenous fluid bag is blocked by the cover whenever the cover is attached to the intravenous fluid bag pole and positioned over the intravenous fluid bag surface opposed to the bag surface displaying information on the contents of the intravenous fluid bag.

17. The cover of claim 16, wherein the front surface is a moldable elastomeric material.

18. A kit for decorating a cover for an intravenous fluid bag comprising:
(a) a cover having an opaque front surface and two side panels attached to opposed edges of the front surface substantially perpendicular to the front surface;
(b) a selectably attachable three sided frame; and
(c) a plate that fits into the three sided frame whenever the frame is mounted on the front surface of the cover.

19. The kit of claim 18 further comprising decorative materials selected from the group consisting of coloring pens, stickers, or photographs.
